(19) United States
(12) Patent Application Publication
(10) Pub. No.: US 2020/0389545 A1
(43) Pub. Date: Dec. 10, 2020

(54) METHODS AND SYSTEMS FOR CONTROL OF THE TRANSMISSION OF MEDICAL IMAGE DATA PACKETS VIA A NETWORK

(71) Applicant: Siemens Healthcare GmbH, Erlangen (DE)

(72) Inventor: Harald Igler, Hausen (DE)

(73) Assignee: Siemens Healthcare GmbH, Erlangen (DE)

(21) Appl. No.: 16/893,711

(22) Filed: Jun. 5, 2020

(30) Foreign Application Priority Data
Jun. 5, 2019 (EP) 19178385.1

(51) Int. Cl. H04L 29/08; H04L 12/861

(52) U.S. Cl. CPC H04L 69/324; G06N 3/08; H04L 49/90

(57) ABSTRACT

A method and device to control the transmission of medical data packets via a network is provided. By accessing a trained model, an optimal point in time for the data transmission is determined, taking into account the respective transmission prerequisites that the data packet has and taking into account the actual network characteristics data. If necessary, the data packet may be buffered for this purpose in a buffer memory.

### CROSS REFERENCE TO RELATED APPLICATIONS

This patent application claims priority to European Patent Application No. 19178385.1, filed Jun. 5, 2019, which is incorporated herein by reference in its entirety.

### BACKGROUND

### Field

The present disclosure relates to the control of the transmission of data packets, such as e.g. high-volume medical image data, via a network from a transmit node to a receive node.

### Related Art

The quantity of data to be transmitted is increasing continuously, in particular in the medical domain. This is due not least to the fact that more and more automatic analysis methods exist and are being applied for the purpose of evaluating the data. For example, more and more clinics are exchanging data so as to be able to ensure better medical care (e.g. by enabling experts working at remote computers to be involved, or by post-processing acquired image data at external computer centers). Moreover, the option of controlling a system remotely, and performing interventional operations e.g. with the aid of camera systems, is to be provided. This requires a stable, secure data flow.

A stable network for secure data transmission is then also essential.

Specifically the transmission of data packets with a high data volume may load the network, which may potentially lead to overloading of the network and occasionally even to malfunctions.

Because the networks in different states and regions of a country are not designed in the same way, malfunctions may occur in practice due to the technical network limitations. Secure transmission of the data then cannot always be ensured.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 illustrates a schematic representation of a control node for control of the transmit operation for a data packet, according to an exemplary embodiment of the disclosure.

FIG. 2 illustrates a control node with further structural units according to an exemplary embodiment of the disclosure.

FIG. 3 illustrates a schematic representation of a modified data packet according to an exemplary embodiment of the disclosure.

FIG. 4 illustrates a schematic representation of a modified data packet according to an exemplary embodiment of the disclosure.

FIG. 5 is a flowchart of a method according to an exemplary embodiment of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

The object underlying the present disclosure is to provide technical means so as to be able still to ensure a reliable and secure transmission of data packets even when the underlying network is limited, at least in phases, in respect of its transmission capacities.

This object is achieved by means of a method, a control node, a computer program and a computer program product.

According to a first aspect, the object is achieved by a computer-implemented method of generating a transmit command set for control of the transmission (in particular the sending) of data packets via a digital network, in which a trained model (such as a trained neural network) is provided, which is stored in a memory and is trained to calculate, for a particular data packet with corresponding transmission prerequisites, and taking into account network characteristics data, a transmit command set such that the transmission prerequisites can be fulfilled when the data packet is transmitted. The method further comprises the following method steps:
Acquiring the data packet to be transmitted;
Calculating transmission prerequisites for the data packet to be transmitted;
Acquiring actual network characteristics data;
Applying the trained model with the acquired transmission prerequisites and the acquired actual network characteristics data in order to calculate and provide the transmit command set.

Aspects of the disclosure relate to an intelligent control of the transmission of the data packets, in which the conditions for the transmission (when should the transmission occur, in which form: the data packet in full or in the form of hashed subpackets, encrypted, using which transmission protocol, etc.) are calculated dynamically and as a function of the actually acquired network conditions (such as network utilization, for example) and by accessing a model in which knowledge about the transmission of data packets is stored. The model may be, for example, a trained neural network.

In one advantageous embodiment of the disclosure, the trained model is used for the application of methods of machine learning. In this context, in particular the transmission durations actually measured are fed back to the trained model in a feedback loop in order to provide a tuning of the model and configure the system so as to be self-learning.

In one advantageous embodiment, the transmit command set comprises a time parameter and/or a hash parameter, wherein the time parameter defines the point in time at which the transmit command is to be executed and wherein the hash parameter defines whether, and if so how, the data packet to be transmitted is to be hashed into subpackets, which are transmitted independently and separately from one another. As a result, the point in time of transmission and/or the manner of transmission of the data packet can be adjusted optimally to the actual network conditions. This measure can ensure that the data transmission is performed reliably even if the network is partially overloaded or cannot provide adequate performance. This is helpful in particular in countries that do not have an adequately designed network.

In one advantageous embodiment, the trained model will have been trained using training data and a supervised learning procedure. In supervised learning, a specific result (transmit command set) for the different input options (transmission prerequisites and network characteristics data) is given. On the basis of the constant comparison between the target and actual results, the network learns to connect the neurons appropriately. The training data may comprise planning data (e.g. time parameters such as duration, feedback on duration, speed parameters or number of faulty packets/items of data), reference data from comparable systems and/or simulation data and/or historical data. With regard to the simulation data, the following should be noted: a clinical network consists of a very wide variety of components (e.g. various scanners (MR and/or CT, ultrasound equipment, etc.), patient data system and billing system). The performance within the clinical network and dependency (susceptibility to malfunction/faults) can be simulated in advance. In this context, in some cases up to 150 network nodes (some of which are DICOM nodes) are to be monitored or included the simulation. This simulation data is taken into account.

In a method according to one or more aspects, in which the data packets to be transmitted are categorized by priority, the priority is determined automatically by the transmit node and in particular by the application of an analysis algorithm to the content of the data packet.

In one advantageous embodiment, the data packets to be transmitted are extended to include an identifier field, wherein the identifier field comprises at least one indication of the priority of the data packet. The priority may be defined by the transmit node. The priority may be calculated automatically by means of a prioritization algorithm. In this context, it may be possible to take into account, for example, whether this is a routine transmission, outage data, or a data packet indicating a total failure of the system. It may then be possible to utilize the available network capacity better.

In a further advantageous embodiment, the data packets to be transmitted are extended to include a request field, wherein the request field comprises an activation function by means of which it may be possible to request the particular data packet from an external receive node. It may then be possible to implement a type of PULL operation, even if under normal conditions a PUSH operation is realized, in which the transmission is triggered from transmit nodes, whereas in PULL operation the transmission is triggered from the receive node.

In the above description, the achievement of the object has been described in relation to the method. Features, advantages or alternative embodiments mentioned in this connection can also be applied equally to the other aspects, and vice versa. In other words, the embodiments (which focus on a control node or a computer program product, for example) can also be developed with the features described in relation to the method. The corresponding functional features of the method are in this case embodied by corresponding concrete modules, in particular by hardware modules or microprocessor modules, of the system or product, and vice versa.

In a further aspect, the disclosure relates to a control node for control of the transmission (in particular the sending) of data packets from a transmit node to a receive node via a digital network. The control node is designed to carry out a method as described above. In this context, the control node may be developed in order to implement the alternative embodiments and/or features of the method described above.

In a further exemplary embodiment of the disclosure, the control node comprises a first interface for acquiring the data packet with the transmission prerequisites and/or a second interface for acquiring actual network characteristics data and/or a third interface for outputting the transmit command set and/or for receiving measurement data actually measured about the data transmission executed. In alternative embodiments of the disclosure, the receipt of data may also be split over further interfaces such that, for example, the data packet is received via a different interface than the transmission prerequisites, if these are not calculated directly in the control node, which is likewise possible. For this purpose, a functionality (e.g. in the form of an algorithm) is provided on the control node, with which functionality the transmission prerequisites are calculated automatically from the data packet, by accessing e.g. historical data and/or by accessing the model.

In a further exemplary embodiment of the disclosure, the control node comprises a model memory. Alternatively, the model memory may be disposed on an external node, which the control node can access, in which external node the trained model is stored.

In a further exemplary embodiment, the control node comprises a memory or can access a memory in which the data packet to be transmitted is buffered before it is sent. This ensures that all data packets to be transmitted are transmitted securely, and that, in the process, the transmission is adjusted and optimized in respect of the actual network conditions.

In a further aspect, the disclosure relates to a computer program product with program code for carrying out a method as described above when the computer program is executed on an electronic device or on a computer.

In a further aspect, the disclosure relates to a computer program with program code for carrying out a method as described above when the computer program is executed on an electronic device or on a computer. In this context, it is also possible for the computer program to be stored on a medium that can be read by a computer, such as e.g. an internal or external memory. The computer program may be configured in a distributed manner such that, for example, individual method steps are carried out on a first computer unit and other method steps are carried out on a second computer unit. In addition, individual method steps may be carried out in a different order. This refers in particular to acquiring the data packet, calculating transmission prerequisites and acquiring the actual network status with the characteristics data. Furthermore, individual method steps may be carried out repeatedly.

One important aspect of the present application is the adaptive adjustment (tuning) of the model. This makes the system self-learning and it can gradually refine and adjust the model using the data collected continuously, and in particular by taking into account the transmission data actually measured (measurement data). Furthermore, external data, e.g. historical data from previous transmissions or data from reference systems, may also be included in the model. Algorithms for the analysis of large volumes of data (big data analysis) may be applied accordingly in the model.

The method is computer-implemented and is executed on a computer unit with a processor. The control node may be realized as, or may comprise, such a computer unit. In an exemplary embodiment, the control node (and/or one or more other nodes described herein) includes processor circuitry that is configured to perform one or more functions and/or operations of the node.

The computer unit may be realized in a machine, e.g. in a computer, in a personal computer, or as a workstation and/or in virtual form in a computer network, and comprises a processor (processing unit) and may comprise a system memory and a system bus that links various system components including the system memory to the processing unit. A processor should be understood in this context to be an electronic circuit, for example. The processor may be, in particular, a Central Processing Unit (CPU), a microprocessor or a microcontroller, for example an Application-Specific Integrated Circuit or a digital signal processor, possibly in combination with a memory unit for storing program commands, etc. A processor may also be, for example, an Integrated Circuit (IC), in particular a Field Programmable Gate Array (FPGA), or an Application-Specific Integrated Circuit (ASIC), or e.g. a multi-chip module, e.g. a 2.5D or 3D multi-chip module, in which in particular multiple ‘dies’ are connected together directly or via an interposer, or a Digital Signal Processor (DSP) or a Graphics Processing Unit (GPU). A processor may also be understood as a virtual processor, a virtual machine or a soft CPU. It may also be, for example, a programmable processor that is equipped with configuration steps to carry out the aforementioned method according to the disclosure, or is configured with configuration steps such that the programmable processor realizes the inventive features of the method, the component, the modules, or other aspects and/or partial aspects of the disclosure.

The computer unit may also contain a hard disk drive for reading from and writing to a hard disk, a magnetic disk drive for reading from or writing to a (for example removable) magnetic disk and an optical disk drive for reading from or writing to a removable (magnetic) optical disk such as a compact disk or other (magnetic) optical media. The hard disk drive, the magnetic disk drive and the (magnetic) optical disk drive may be linked to the system bus via a hard disk interface, a magnetic disk drive interface and a (magnetic) optical drive interface. The drives and associated storage media provide non-volatile storage for machine-readable instructions, data structures, program modules and other data for the computer. Although the exemplary environment described here uses a hard disk, an exchangeable magnetic disk and an exchangeable (magnetic) optical disk, the person skilled in the art will know that other types of storage media, such as e.g. flash memory cards, Random-Access Memory (RAM), Read-Only Memory (ROM) and similar, may be used instead of or in addition to the storage facilities presented above. A basic input/output system (BIOS) containing basic routines that help to transfer information between elements within the PC, e.g. during startup, may be stored in the ROM. Multiple program modules, such as an operating system, one or more application programs, such as the method of calculating a transmit command set, and/or other program modules and/or program data for example, may be stored on the hard disk, the magnetic disk, the (magnetic) optical disk, the ROM or the RAM. A user may input commands and information into the computer using input apparatuses, such as a keyboard and a pointing device, for example. Other input apparatuses such as microphone, satellite dish, scanner or similar may also be included. These and other input devices are often connected to the processing unit via a serial interface linked to the system bus. However, input devices may also be connected via other interfaces, for example via a parallel interface or a Universal Serial Bus (USB). A monitor (e.g. a GUI) or another type of display apparatus may also be connected to the system bus via an interface such as e.g. a video adapter. In addition to the monitor, the computer may also include other peripheral output devices such as loudspeaker and printer.

The computer unit is operated in a network environment or “network” that defines logical connections to one or more remote computers (nodes). The remote computer may be another personal computer, a server, a router, a network PC, a peer device or another common network node, and may contain many or all of the elements described above in relation to the personal computer. The logical connections may include a Local Area Network (LAN) and a Wide Area Network (WAN), an intranet and the Internet.

A “memory” or a “memory unit” or a “memory module” and the like may be understood in connection with the disclosure as, for example, a volatile memory in the form of Random-Access Memory (RAM) or a permanent memory such as a hard disk or a data medium or e.g. a removable memory module.

A “node” (transmit node, receive node) may be understood in connection with the disclosure as, for example, a computer unit (as described above), a processor and/or a memory unit for storing program commands. For example, the processor is specifically configured to execute the program commands such that the processor carries out functions in order to implement or realize the method according to the disclosure or a step of the method according to the disclosure. The nodes are configured with corresponding network interfaces. In an exemplary embodiment, one or more nodes include processor circuitry that is configured to perform one or more functions and/or operations of the respective node(s).

“Providing”, in particular in relation to the transmit command (data) set, may be understood in connection with the disclosure as, for example, providing with the aid of a computer. Providing is performed for example via an interface (e.g. a database interface, a network interface, an interface to a memory unit). During providing, via this interface corresponding data and/or information may be transferred and/or sent and/or called up and/or received e.g. to external electronic entities. A communication module, such as e.g. an Ethernet interface or network card, may be made available for this purpose.

The above configurations and developments can be combined with one another as desired, wherever useful. Further possible configurations, developments and implementations of the disclosure also include not explicitly mentioned combinations of features of the disclosure described above or in the following in relation to the exemplary embodiments. In particular, in this context the person skilled in the art will also add individual aspects as improvements or enhancements to the respective basic form of the disclosure.

The term “transmit command data set” is a digital data set that may comprise multiple elements. It is used for control of the transmit operation and comprises, in essence, the transmit command for the data packet. In addition, it may set out further specific technical details in respect of the transmit operation. Which technical parameters are to be taken into account in this context may be configurable in an exemplary embodiment of the disclosure. In an exemplary embodiment, the transmit command data set includes a point in time of transmission and the manner of transmission (monolithically or as hashed components).

The term “transmission prerequisite” means the technical preconditions that must be fulfilled for the transmission of the data packet via the network so that a secure and reliable transmission can be ensured. These may include e.g. in particular a minimum bandwidth, number of intermediary nodes, use of encryption technology, maximum permissible duration of the transmission and/or a point in time or a time phase of the transmission. Further examples are an inflow and outflow of the data, a period of time from command transmission to execution, and feedback, if applicable.

The term “network characteristics data” refers to the technical properties and features of the network. These are typically variable over time. They may include in particular a minimum and maximum bandwidth, an operating mode of the network and/or a maximum utilization. The operating mode of a network includes, inter alia, the network topology, such as a ring network or a linear network. For example, in the event of an outage, a ring network in a clinic system may be able intelligently to select another transmission path. The external transmission could be performed e.g. in a linear network with a certain architecture. In addition, further characteristics data may be derived from the network characteristics data. For example, congestion may be present in a 10 Mbit/s or 100 Mbit/s cable, or the performance of a cable may deteriorate because an optocoupler becomes weak or a cable connection loosens. However, it is also possible for the data to be transmitted for example via a radio network/satellite network and exposed to further influences there (weather, condition of the transmitter masts, etc.). The actual status of these variables, which vary over time, is acquired and they are used for the calculation.

The trained model is a computer model that models the network traffic with its technical parameters, such as e.g. a minimum, maximum, and available bandwidth. In this context, “trained” means that, on the basis of prior knowledge in the form of training data, the network has been trained for a certain task, in particular for the calculation of command data for initiating the data transmission of a data packet, so that its transmission prerequisites can be adhered to under the prevailing network conditions or given the prevailing network status determined with the aid of the network characteristics data. The training data also comprises the actual transmission conditions, in particular the actual transmission time. In this way, a link can be learned between (actually required or resulting) transmission time for a data packet with certain transmission prerequisites given certain network characteristics data. A sufficient volume of training data allows a learning model to be generated. With the aid of the learning model, it is possible to generate a transmit command set that specifies in particular when the data packet ideally should be transmitted under the actual network conditions (characteristics data) in order that the transmission prerequisites can reliably be adhered to.

The model is configured to generate the transmit command set such that, when the data packet is sent, the network load is as low as possible and/or the transmission prerequisites acquired can reliably be adhered to (which may also include minimum network capacity, for example). For generating the transmit command set, the model takes into account the actual status of the network with preconfigurable parameters, such as historical data, network utilization, peak network loads, etc. Furthermore, the model is also designed to provide predictions about the future network load based on the data collected and acquired. In this way, the model may also be applied as part of predictive maintenance measures to the monitoring of network quality and/or to the nodes connected to it and may include predictive data. For example: if, on the basis of analyzing historical data, it can be inferred that a network status will be achieved imminently (e.g. overnight) that permits a higher bandwidth, the transmission of the data packets may be scheduled for that period of time. As a result, the technical advantage may be achieved that a reliable and secure transmission can be ensured, even in the case of networks with a lower transmission capacity than that stipulated by the transmission prerequisites, by the model generating a transmit command set such that it defines that the data packet is actuated only in times of low utilization (and a sufficient data transfer rate) and/or in hashed and/or compressed form, namely hashed into smaller subpackets. Furthermore, malfunctions may then be calculated in advance and beforehand and deployed e.g. as part of predictive maintenance. As a result, physical faults in the network (e.g. hardware problems, lost connection) may be indirectly detected automatically and reported. Overall, an intelligent adaptation to network fluctuations (improvement and deterioration of bandwidth) is possible.

The model may be a trained neural network or another artificial intelligence (AI) module. In this aspect, the AI module or the model supply manipulated variables with which the command to transmit the data packet can be generated or configured such that a specified transmission prerequisite is met. In an exemplary embodiment, AI modules are trained primarily with empirical knowledge from historical, recorded data transmissions or other empirical data or simulation data. Here, the input neurons comprise the data packet itself, its transmission prerequisites and the actual network conditions (characteristics data). The output neurons supply the transmit command set with a point in time (or time phase) of transmission, and optionally also further transmission parameters. The architecture of the network depends on the level of detail with which the transmit command set is to be calculated. In an exemplary embodiment of the disclosure, a recurrent network is applied. Recurrent networks also have reverse (recurrent) edges (feedback loops) and thus include feedback. Edges of this type are then often provided with a time delay, so that, in step-by-step processing, the neuron outputs of the previous unit can be reapplied as inputs. These feedback loops allow a network to behave dynamically and thus equip it with a type of memory. Furthermore, a convolutional neural network (CNN) and/or a network with additional intermediate layers may be used.

The acquisition of the network characteristics data may be performed by means of a data collector (sniffer). In an exemplary embodiment, in an upstream configuration phase, it may be possible in this context to configure which characteristics data is to be acquired.

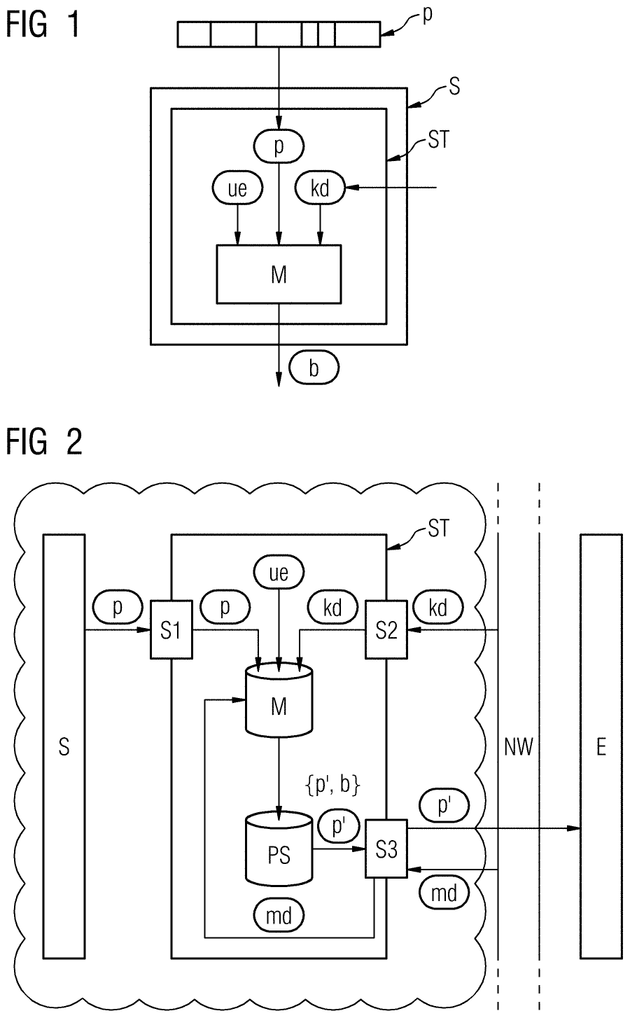

FIG. 1 shows a high-level schematic overview of a computer-implemented control node ST, which may be implemented on a transmit node S and analyzes the data traffic between the transmit node S and one or more receive nodes E in order to control the data transmission. As indicated in FIG. 1, a data packet p or a quantity of data packets is/are to be transferred from the transmit node S to the at least one receive node E. To this end, the data packet p is transferred firstly, and before the transmission, to the control node ST. In an exemplary embodiment, since the control node ST is disposed in the transmit node, no access to the network is necessary for this purpose. The control node ST can calculate the transmission prerequisites from the data packet p to be transmitted. To this end, it may access e.g. a rule base or a memory. Alternatively, it is possible for the transmission prerequisites ue to be transferred with the data packet p and for the control node ST not to have to calculate these. Furthermore, characteristics data kd is acquired from the network NW, by means of which the data packet p is to be sent to the receive node E. All acquired or calculated data p, ue, kd is supplied to a trained model M, which is configured to calculate a transmit command set b in response to the values supplied. The transmit command set b may comprise multiple fields and specifies an optimal point in time or such a time phase that is most suitable for the purpose of transmitting the data packet via the network NW so that its transmission prerequisites ue can be adhered to.

FIG. 2 shows further components of the nodes involved. The control node is configured here as a separate node, but which exchanges data with the transmit node via a high-performance network. This is intended to be indicated in FIG. 2 by the curved border around both nodes S, ST. A first interface S1 is made available for acquiring the data packet p. The acquisition of the network characteristics data kd is performed via a second interface S2. The transmission prerequisites ue may be transferred to the control node, e.g. likewise via the first interface with the data packet p, or the transmission prerequisites ue may be calculated locally on the control node ST, possibly by accessing a memory and/or by means of an algorithm on the basis of the acquired data packet p. This data p, ue, kd is supplied to the trained model M, which then generates the transmit command set b. Because the transmit command set b actuates the data transmission of the data packet p possibly only at a later point in time, the data or data packet p must be buffered. In an exemplary embodiment, a buffer memory PS is disposed for this purpose in the control node ST and in the transmit node S.

In an exemplary embodiment of the disclosure, a modified data packet p′ may be generated by the application of the model M. The modified data packet p′ may be extended compared with the (original) data packet p. In particular, an identifier field fd and/or a request field of may be configured.

The identifier field kf is used for storage of a calculated priority of the data packet p. Said priority may be represented numerically or in preconfigurable classes and may define e.g. a high priority if the data packet p represents total failure data and e.g. a low priority if the data packet p relates to a routine transmission that will be repeated anyway. By acquiring this parameter (priority), the model M may identify patterns of priority and use these for future calculations.

The request field af is used for storage of an activation function by means of which an external node, e.g. the receive node E, is enabled specifically to request the data packet p.

In an exemplary embodiment, the actual result of the transmission of the data packet p, e.g. in the form of the transmission time actually required, is measured. Further measurement data md may also be determined, such as e.g. a quality of the transmission, etc. This measurement data md is then fed back to the control node ST and may be supplied to the model M in a feedback loop. This is represented schematically in FIG. 2 by the arrow marked with the reference character md. As a result the model may be configured so as to be adaptive and self-learning. In this way, in particular the weightings between the neurons in the network may be modified, for example, if the actual measurement data md indicates that the requested transmission prerequisites ue could not be adhered to a satisfactory extent. In an exemplary embodiment, the model M is automatically adapted based on the measurement data.

The model may have been trained using training data and/or using reference data from other transmit node-network-receive node systems and/or using historical data.

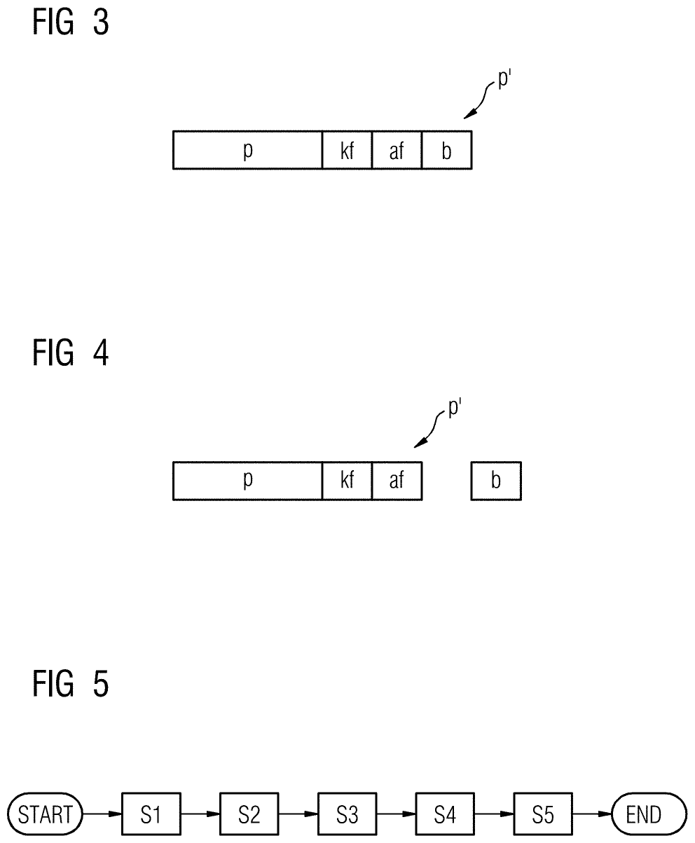

This is shown schematically in FIG. 3. The modified data packet p′ comprises the calculated transmit command set b and may also have the identifier field kf and the request field af in addition to the data packet p. Alternatively, it is possible for the original data packet p to be stored separately.

FIG. 4 shows an alternative embodiment. Here, the identifier field kf and/or the request field af is/are appended to the original data packet p, while the transmit command set b is stored separately in a data structure that is assigned to the respective other component, namely the modified data packet p′.

FIG. 5 shows a flow chart of a control method according to an exemplary embodiment of the disclosure. After the method is started, in step S1 the data packet p to be transmitted is acquired. In step S2 the transmission prerequisites ue for the respective data packet p are determined. This may be performed either by means of a calculation—e.g. by accessing a memory and applying an automatic calculation method—or the transmission prerequisites ue are acquired directly with the data packet p. In step S3, the actual characteristics data kd of the network NW, via which the data packet p is to be transferred, is acquired and may then be analyzed on the control node ST. All variables p, ue, kd provided are then supplied to the model M, which calculates in step S4 the transmit command set b for this data and provides it in step S5, and in particular in this context specifies a point in time for a transmission. In order to prepare the transmission, the data packet p is buffered in the buffer memory PS. The method may then be applied for the next data packet p or—e.g. in the event of a fault—repeated before the method ends.

Finally, it should be noted that the description of the disclosure and the exemplary embodiments should not be seen as in any way restrictive with regard to a particular physical realization of the disclosure. All features shown and explained in conjunction with individual embodiments of the disclosure can be made available in a different combination in the inventive subject matter in order at the same time to realize its advantageous effects.

The scope of protection of the present disclosure is provided by the following claims and is not restricted by the features explained in the description or shown in the figures.

For a person skilled in the art, it is obvious, in particular, that the disclosure may be applied not merely to medical image data, but also to other medical data. Likewise, it may be applied to data packets that are present in home networks (SmartHome) or in power networks and have to be transmitted via a corresponding network. Furthermore, the components of the control node ST may be realized in a distributed manner on multiple physical products.

Any connection or coupling between functional blocks, devices, components of physical or functional units shown in the drawings and described hereinafter may be implemented by an indirect connection or coupling. A coupling between components may be established over a wired or wireless connection. Functional blocks may be implemented in hardware, software, firmware, or a combination thereof.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processor circuitry” shall be understood to be circuit(s), processor(s), logic, or a combination thereof. A circuit includes an analog circuit, a digital circuit, state machine logic, data processing circuit, other structural electronic hardware, or a combination thereof.

A processor includes a microprocessor, a digital signal processor (DSP), central processor (CPU), a microprocessor or a microcontroller, application-specific instruction set processor (ASIP), graphics and/or image processor, multi-core processor, or other hardware processor. A processor may also be, for example, an Integrated Circuit (IC), such as a Field Programmable Gate Array (FPGA), or an Application-Specific Integrated Circuit (ASIC), or e.g. a multi-chip module, e.g. a 2.5D or 3D multi-chip module, in which in particular multiple ‘dies’ are connected together directly or via an interposer, or a Digital Signal Processor (DSP) or a Graphics Processing Unit (GPU). A processor may also be understood as a virtual processor, a virtual machine or a soft CPU. It may also be, for example, a programmable processor that is equipped with configuration steps to carry out the methods according to the disclosure, or is configured with configuration steps such that the programmable processor realizes the inventive features of the method, the component, the modules, or other aspects and/or partial aspects of the disclosure.

The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A computer-implemented method of generating a transmit command set for control of the transmission of data packets via a digital network, in which a trained model is provided, which is stored in a memory and is trained to calculate, for a data packet with corresponding transmission prerequisites, and taking into account network characteristics data, the transmit command set such that the transmission prerequisites are fulfilled when the data packet is transmitted, the method comprising:
acquiring the data packet to be transmitted;
calculating transmission prerequisites for the data packet to be transmitted;
acquiring actual network characteristics data associated with the digital network; and
applying the trained model with the calculated transmission prerequisites and the acquired actual network characteristics data to calculate and provide the transmit command set.

2. The method as claimed in claim 1, wherein the trained model is configured for machine learning, measurement data and measured transmission durations being fed back to the trained model in a feedback loop.

3. The method as claimed in claim 2, wherein the trained model is automatically adapted based on the measurement data and/or measured transmission durations fed back to the trained model.

4. The method as claimed in claim 1, wherein the trained model is a neural network.

5. The method as claimed in claim 1, wherein the transmit command set comprises a time parameter and/or a hash parameter, the time parameter defining a point in time at which a transmit command is to be executed, and the hash parameter defining whether, and if so, how, the data packet to be transmitted is to be hashed into subpackets, wherein the subpackets are transmitted independently and separately from one another.

6. The method as claimed in claim 1, wherein the trained model has been trained with training data and a supervised learning process.

7. The method as claimed claim 6, wherein the training data comprises reference data, planning data and/or simulation data.

8. The method as claimed in claim 1, further comprising categorizing the data packets to be transmitted by priority, wherein the priority is determined automatically by the transmit node by an analysis algorithm applied to respective content of the data packets.

9. The method as claimed in claim 8, wherein the data packets to be transmitted are extended to include an identifier field that includes at least one indication of the respective priority of the data packets.

10. The method as claimed in claim 1, further comprising extending the data packets to be transmitted to include a request field that includes an activation function configured to facilitate a request of the particular data packet from an external receiver node.

11. A computer program product having a computer program which is directly loadable into a memory of a computer, when executed by the computer, causes the computer to perform the method as claimed in claim 1.

12. A non-transitory computer-readable storage medium with an executable program stored thereon, that when executed, instructs a processor to perform the method of claim 1.

13. A control node for control of transmission of data packets from a transmit node to a receive node via a digital network, comprising:
a processor configured to:
acquire a data packet to be transmitted;
calculate transmission prerequisites for the data packet to be transmitted;
acquire actual network characteristics data associated with the digital network; and
apply a trained model with the calculated transmission prerequisites and the acquired actual network characteristics data to calculate and generate a transmit command set to control of the transmission of data packets, wherein the transmission prerequisites are fulfilled when the data packet is transmitted.

14. The control node as claimed in claim 13, further comprising:
a first interface configured to acquire the transmission prerequisites;
a second interface configured to acquire the actual network characteristics data; and
a third interface configured to output the generated transmit command set and/or to receive measurement data associated with the data packet transmission.

15. The control node as claimed in claim 13, further comprising a memory that stores the trained model, the processor being configured to access the memory.

16. The control node as claimed in claim 13, wherein the processor is configured to access a memory that stores the trained model.

17. The control node as claimed in claim 13, further comprising: a buffer memory configured to buffer the data packet to be transmitted before the data packet is sent.

18. The control node as claimed in claim 13, wherein the processor is configured to access a buffer memory that buffers the data packet to be transmitted before the data packet is sent.
